(19) United States
(12) Patent Application Publication
(10) Pub. No.: US 2005/0213034 A1
(43) Pub. Date: Sep. 29, 2005

(54) Eye training equipment

(76) Inventor: Katsumi Nagayoshi, Tokyo (JP)

Correspondence Address: GREENBLUM & BERNSTEIN, P.L.C., 1950 ROLAND CLARKE PLACE, RESTON, VA, 20191, US

(21) Appl. No.: 10/510,950

(22) PCT Filed: Apr. 24, 2003
(86) PCT No.: PCT/JP03/05250

§ 371 Date: Oct. 28, 2004

(30) Foreign Application Priority Data
Apr. 30, 2002 (JP) 2002-128083

(51) Int. Cl. A61B 3/00

(52) U.S. Cl. 351/203

(57) ABSTRACT

Eye training equipment, wherein at least two irritation generating devices for perceiving irritating positions are disposed around an eye ball, and the disposed irritation generating devices are operated one by one to allow the line of sight of a user to follow up in the direction of the irritating position, whereby, since merely the line of sight of the user is allowed to follow up in the direction of the irritating position, the user of the training equipment can perform an eye muscle motion without requiring the need for specifically viewing an object by opening an eye.

### TECHNICAL FIELD

The present invention relates to an eye training equipment usable for aiding a user in regaining his or her eye-muscular strength through active exercise of the eye muscles by having the user keep his eyes on a moving target as well as in gaining an improved kinetic visual field which is very important especially in playing sports by training his eye muscles.

### BACKGROUND ART

Using the well-known eye-muscle training method or equipment requires the user or trainee to keep his eyes on a moving object as a target. Also, keeping eyes on the moving fingers while winding one arm before a user by himself is known to be highly effective for training the eye muscles.

Actually, however, the arm winding needs a space where at least the arm can be wound, and will cause the user to feel as if the scene surrounding him and the room internal scene were rotating, which will bring a dizziness to the user having made only a few minutes of such a training. Therefore, it will be dangerous for any aged person having declined in sense of balance to try this training in upright position outdoors.

Many goggle-type eye training devices have been developed so far, and some of them are commercially available. Use of such a goggle-type device needs no wide space, and the user will not have to move his arm, will not feel fatigued by the training and can make the training in sitting on a chair or the like that or lying down on the bed. Thus, this type of eye training device is improved in safety without any danger such as a dizziness-caused falling. However, with all the conventional eye training devices of the goggle type, the user has to actually keep his eyes on a light spot such as an image or light-emitting diode. A user having near-sighted eyes will be able to clearly see any very near image or light spot. However, a far-sighted user or a presbyopic user who could see over a long distance in his youth will hardly be able to clearly see the very near image or light spot. For training the eye muscles, the presbyopic user has to put on a pair of glasses. Otherwise, such a presbyopic user cannot clearly see a very near target with his own eyeglasses used in the daily life. The eye training itself with such a conventional goggle-type eye training device will be very hard to such a user. The user will not be able to be patient even with a basic training pattern in which he has to try a few minutes of such an eye training routinely everyday. Also with the well-known eye training to be done with concentration on having a definite view of a concrete target such as an image, light spot, letter, symbol or the like, the user will blink an extremely reduced number of times for the period of training, which will cause the user to have a dry-eye condition in his eyes. Therefore, the eye training with the conventional eye training device will be very hard to the user or trainee.

Generally, it can be said that the persons in their thirties will start being presbyopic and those in their forties will have an accelerated progress of the presbyopic state. The old sight or presbyopic state will come to the human being physically aged. Actually, however, some of the people can keep their eyesight unchanged. Except for the latter, most of the people in fifties will become presbyopic. Generally, even the people having had an eyesight of 1.5 to 2.0 in their youth and who have become far-sighted will hardly be able to focus on an object at a very near distance of 20 cm from the eyes and will be almost unable to read small letters and so on in directions printed on the package of a nonprescription drug.

### DISCLOSURE OF THE INVENTION

Accordingly, the present invention has an object to overcome the above-mentioned drawbacks of the conventional art by providing an eye training equipment usable for aiding a user in regaining his eye-muscular strength through active exercise of the eye muscles by having the user keep his eyes on a moving target as well as in gaining an improved kinetic visual field which is very important especially in playing sports by training his eye muscles, without bringing any discomfort such as “annoyance by inability to clearly view a target, difficulty of keeping eyes on a target or the like” to even a presbyopic or far-sighted user who cannot focus on a very near object with naked eyes.

The above object can be attained by the eye training equipment according to the present invention. This eye training equipment can be used even with the eyelids being closed. So, with the eye training equipment, even a user being so presbyopic or far-sighted as not to be able to focus on a very-near point with naked eyes or who has a difficulty of looking at a target because of a difference between the right and left eyesight can turn his line of vision toward a moving target without bringing, to him, any discomfort such as “annoyance by inability to clearly view a target, difficulty of keeping eyes on a target or the like”. With this eye training equipment, the user can have his eye muscles put into motion just by trying to view in directions from which irritations are sequentially given. So, the eye training with this equipment will be less weighty than the conventional art that requires the user to actually look at a target object and hence the user will be able to have the eye training routinely everyday without overtaxing himself. In the conventional eye training equipment with which the user has to actually look at an object, the viewing angle is on the order of 60 degrees at maximum whether the equipment is of the screen type, monitor type or goggle type. With the eye training equipment according to the present invention, however, the user is irritated alternately nearly at the right and left temples, for example. When the user turns the line of vision to try to view the sources of irritations alternately, the viewing angle will be approximately 180 degrees, which will correspondingly increase the motion of the eyeballs and will thus result in a remarkable improvement in effect of the eye training. As a result, it is possible to recover the eye-muscular strength from the level in the presbyopic condition, delay the aging of the eye muscles, improve the kinetic visual field which is very important especially for the athletes. Namely, the eye training with the equipment according to the present invention will provide a significant effect for the health. Also, since the eye training equipment according to the present invention permits the user to try the eye training even with his eyes being closed, the eyeball surface will not be dry even if the user is devoted to the training. That is, a training for a certain long time will not cause a so-called dry-eye state in which the user will feel pains in the eyes.

### BRIEF DESCRIPTION OF THE DRAWINGS

FIG. 1 is a perspective rear view, given for reference, of a goggle-type eye embodiment of the training equipment according to the present invention.

FIG. 2 is also a perspective rear view, given for reference, of a sunglass-type embodiment of the eye training equipment according to the present invention.

FIG. 3 is a schematic illustration showing an arrangement of contact pieces provided at 4 places.

FIG. 4 is also a schematic illustration showing an arrangement of contact pieces provided at 6 places.

FIG. 5 is a schematic illustration showing an arrangement of contact pieces provided at 12 places.

FIG. 6 is a schematic sectional view of a contact piece having a pressure generator provided therein.

FIG. 7 is a schematic sectional view of a contact piece having a vibration generator provided therein.

FIG. 8 is a schematic sectional view of a contact piece having a heater provided therein.

### BEST MODE FOR CARRYING OUT THE INVENTION

The present invention will be illustrated and described in detail below with reference to the accompanying drawings.

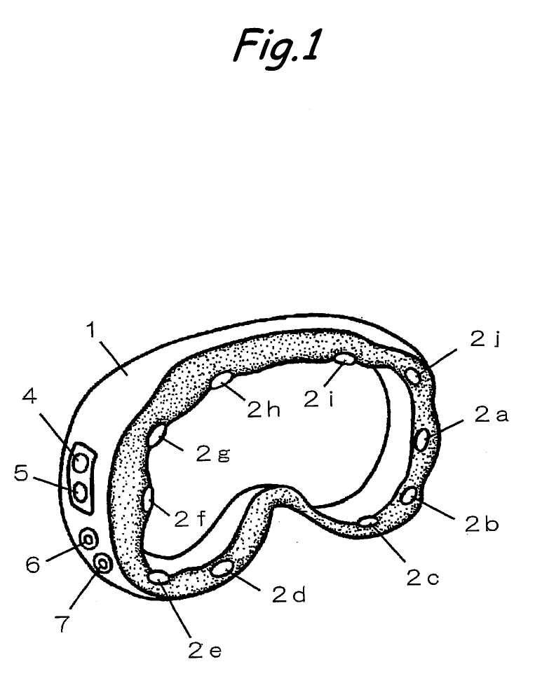

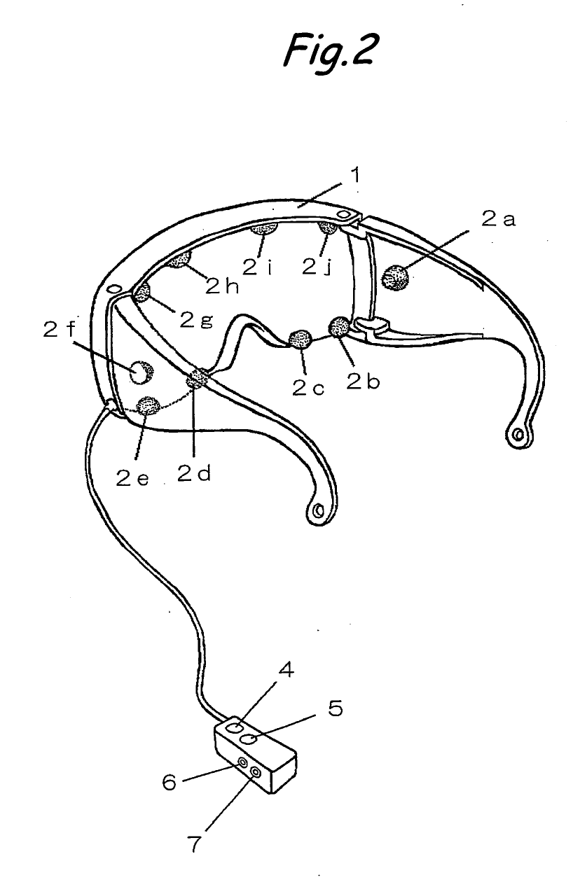

As shown FIG. 1, the eye training equipment according to the present invention includes mainly a goggle-shaped body 1 and a soft face-contact member 3 provided with contact pieces 2a to 2j which are to be put into touch with the user face. The body 1 has installed thereto a power on/off switch 4 and a mode select switch 5 for selecting a user-desired one of modes of operation. The soft face-contact member 3 is formed from a soft rubber, foamed rubber, soft foamed urethane or the like. In case the eye training equipment is designed to be externally powered via an adapter, a power reception jack 6 is additionally provided on the body 1 of the eye training equipment. In case a battery is used as a power source within the body 1, however, the power reception jack 6 is not necessary. Generally, there is provided in the body 1 a main controller in order to allow irritation sources provided within the contact pieces, respectively, to operate sequentially under a program. However, in case a music or a training voice guidance like “Please wind the arm more speedily, this time” or “Now, let's take a pause of 2 minutes. You will hear a soft music. Relax yourself with your eyes being closed” is to be given to the user through a headphone, a personal computer or a dedicated external apparatus will be used in connection with the eye training equipment. In such a case, a control jack 7 may additionally be provided on the eye training equipment and the main controller will be provided outside the eye training equipment. Also, in case an eye training is done under the supervision of an eye trainer according to the user's personal eye condition, 10 contact pieces may be provided in the eye training equipment as shown in FIG. 1 and there may be used a manually-operable 10-direction joystick designed for more than two switches thereof not to be turned on simultaneously. The joystick can effectively be used to allow the user to feel an irritation. In this case, each switch of the joystick is connected in one-to-one relation directly to the irritation source in each contact piece, and any controller operated under a special program will not be required. FIG. 2 is a reference drawing showing a sunglass-type embodiment of the eye training equipment of the present invention, in which a contact piece is provided at each of the right and left face portions (near the temples) adjacent to the respective eyes. It should be noted that all the reference numerals used in FIG. 2 correspond to those in FIG. 1 but that the power on/off switch 4, mode select switch 5, power reception jack 6 and control jack 7 are provided separately from the body 1 as will be seen.

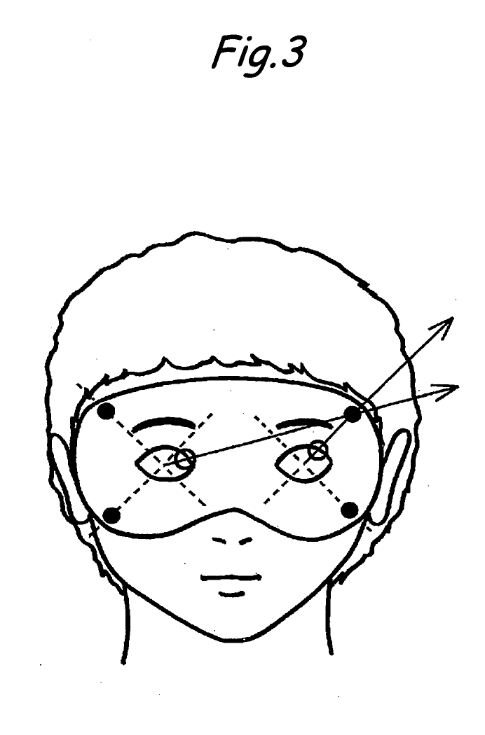

The present invention will be illustrated and described in detail concerning the contact pieces with reference to FIGS. 3 to 5. Each of the drawings schematically illustrates the eye training equipment according to the present invention, which is to be put on the face of a user. It should be noted that each black small circle in the drawings indicates a contact piece having an irritation source provided therein and which is to be into touch with the user's face.

Contact pieces provided at 4 places as shown in FIG. 3 allows the user to direct his lines of vision to toward each of the 4 contact pieces. With this embodiment, the user's eyeballs will be turned in a narrow range but at least horizontally and vertically. The eye muscles can thus be trained to a satisfaction (like a light jogging). From the standpoint of a reasonable eye-muscular training for persons of middle to old age, this embodiment is suitable for use such users to prevent their old sight from progressing. Also, this embodiment can be produced most inexpensively because it uses the minimum number of contact pieces.

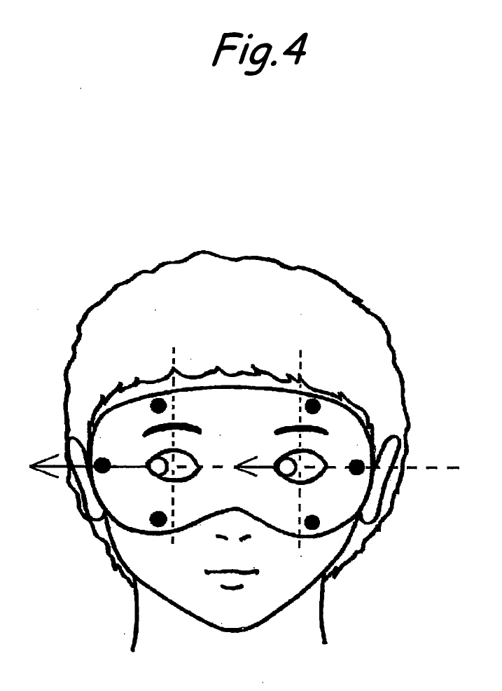

Contact pieces provided 6 places as shown in FIG. 4 permit the user to turn his right and left eyeballs in the horizontal, vertical and oblique directions to a nearly satisfactory extent. With this embodiment, it is possible to resolve a general lack of eye-muscular exercise. The embodiment is satisfactory for improving the blood circulation to prevent the eye muscles from aging. Since the number of contact pieces is larger than that (four) in the embodiment shown in FIG. 2, so this embodiment is more expensive than that shown in FIG. 2, but more effective for the slight cost increase.

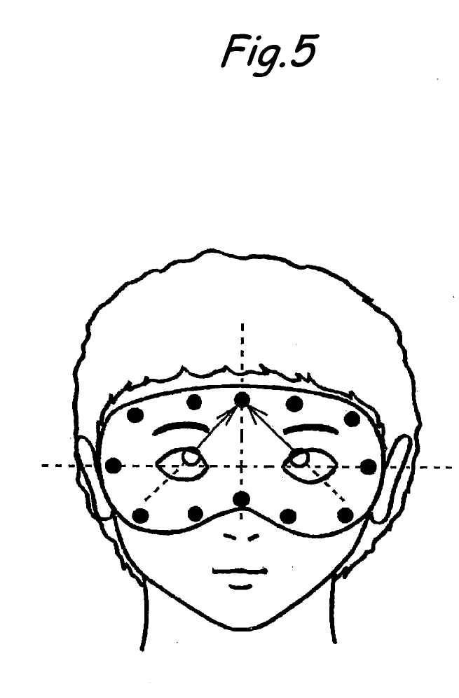

Contact pieces provided at 12 places as shown in FIG. 5 permit the user to turn his right and left eyeballs in the horizontal, vertical and oblique directions to a nearly complete extent. Since the turning speed of the eyeballs can thus be increased, this embodiment is most desirable for use to train the eye muscles of the athletes whose have to direct this lines of vision accurately, rather than for use to prevent the eye muscles from aging.

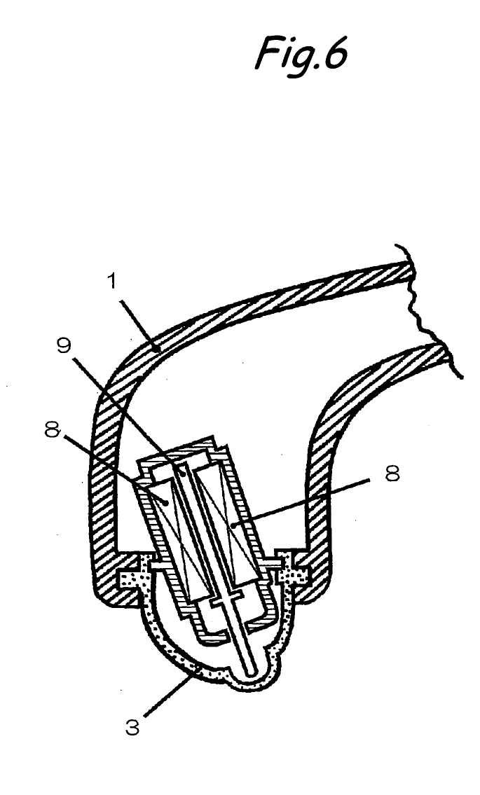

FIG. 6 is a schematic sectional view of an embodiment using a pressure generator as the irritation source. The pressure generator includes an electromagnet 8 formed from coil wound about a magnet rod 9. The magnet rod 9 passes from one side of the electromagnet 8 inside the body 1 to the other side. When the electromagnet 8 is supplied with a current, it will move the magnet rod 9 in a direction in which it will push the soft face-contact member 3. This movement will be an irritation to the user's face. The user will thus recognize the irritation source or irritating position. It should be noted that to force the rod-like member to the soft face-contact member 3 from inside, there may be used a motor and cam combination may be used or a piston and cylinder combination which uses a fluid such as air, liquid or the like. Also, a small balloon-like device may be provided in each irritating position and a fluid such as air, liquid or the like be used to produce a pressure. Although the magnet rod 9 as a pusher is provided inside the soft face-contact member 3 as shown in FIG. 6, it may be designed to have a free end which penetrates out of the soft face-contact member 3 and touches directly the face skin in case a pressure applied has to be recognized precisely as a pin point by the user. The face-contact end (free end) of the magnet rod 9 may be formed from a soft brush or rubber.

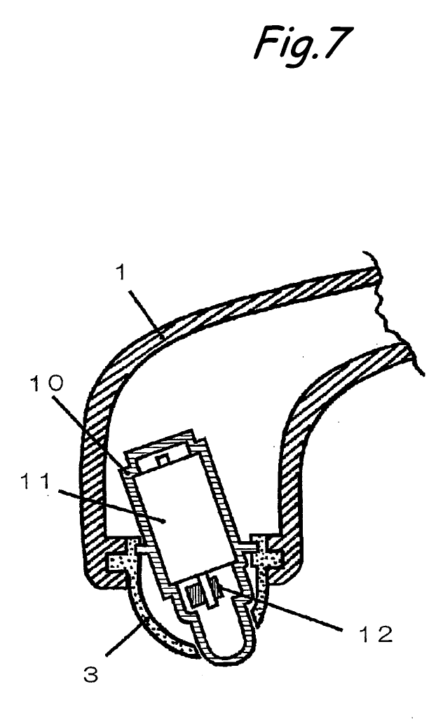

FIG. 7 is a schematic sectional view of another embodiment using a vibration generator as the irritation source. This embodiment includes a housing 10 provided inside the body 1 with allowance of some vibration. In the housing 10, there is set an ultra-miniature motor 11 with an eccentric weight 12. When the ultra-miniature motor 11 is put into run, the eccentric weight 12 will vibrate the motor housing 10. This vibration will be an irritation to the user's face, by which the user will recognize the irritating position. The vibration generator may be an electromagnetic ultra-miniature buzzer or an electromagnetic speaker. A vibration generated by such an ultra-miniature buzzer or speaker may be amplified and transmitted with the use of a cylindrical or generally conical auxiliary structure. By disposing the vibration generator for a part thereof to be exposed outside the soft face-contact member 3, the user will be able to recognize the irritating position more accurately.

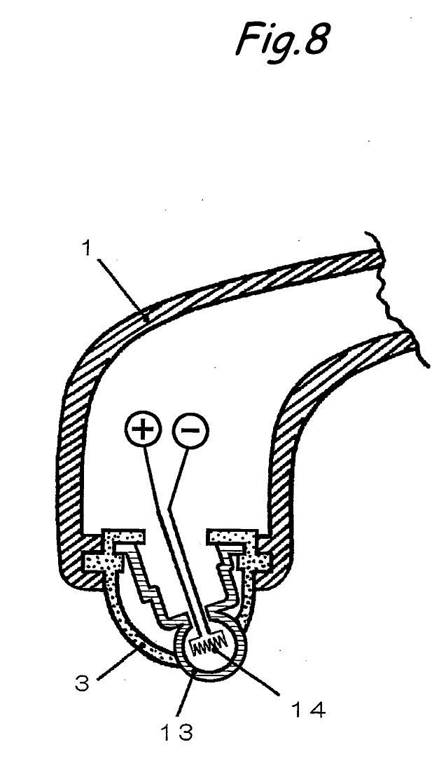

FIG. 8 is a schematic sectional view of a still another embodiment using a heater as the irritation source. The encircled symbols “+” and “−” shown in FIG. 8 indicate that conductors connected to a power source. This embodiment includes a housing 13 provided inside the body 1 and having one end thereof partially exposed outside the soft face-contact member 3. A heater 14 is set inside the housing 13. When the heater is supplied with a current, it will generate a heat which will be conducted to the user's face skin. The housing 13 should preferably be formed from a metal from the standpoint of heat conduction. However, since a metallic housing will cause some discomfort in the beginning of using the eye training equipment, so the face-contact portion thereof should preferably be flocked. In FIG. 8, the heater is a one formed like a small bulb, which will be easiest to understand. Normally, however, the actual heater is a bar-like or flat one to assure the safety and durability. The most significant feature of the embodiment in which a heater is used as the irritation source is that it generates neither noise nor vibration, while the embodiments using the pressure generator and vibration generator, respectively, as the irritation source generate noise and vibration, respectively, which will possibly disturb the user in training his eye muscles. Therefore, the embodiment using the heater as the irritation source can be used without disturbance to nearby people and also while listening to the training guidance and music. Also, the heater provides a temperature output of 38 to 42° C. and can appropriately irritate around the eyes, which will be comfort to the user.

Further, the irritation source may be a weak-current generator. An embodiment using such a weak-current generator is not illustrated herein but it has an apparent basic design similar to the heater as shown in FIG. 8. It should be noted that each of the contact pieces should use water or gel for an improved electrical conductivity as the case may be.

The present invention is not limited to the aforementioned embodiments but may be modified in number, shape and material of the parts. It should be noted that since the present invention relates mainly to mounting of the irritation sources, so the power source, wiring, control system, etc. are not illustrated and explained herein.

### INDUSTRIAL APPLICABILITY

As having been described in the foregoing, the eye training equipment according to the present invention is used just as placed over the eyes. Even a person whose old sight is such that he cannot focus on a very-near point with naked eyes or who has a difficulty of looking at a target because of a difference between the right and left eyesight is allowed to direct his line of vision toward a moving target to turn the eyeballs actively without feeling discomfort and can thus regain his eyesight. Further, with this eye training equipment, the athlete can have his muscles trained to have an improved kinetic visual field.

1. An eye training equipment including two or more irritation generators provided around user's eyes to allow the user to perceive the irritating positions, wherein:
the irritation generators are activated one by one to have the user turn his line of vision toward an irritating position, so that the user can train his eye muscles without actually viewing any target object with the eyes being opened.

2. The equipment according to claim 1, wherein the irritation generator is a pressure generator.

3. The equipment according to claim 1, wherein the irritation generator is a vibration generator.

4. The equipment according to claim 1, wherein the irritation generator is a heater.

5. The equipment according to claim 1, wherein the irritation generator is a weak-current generator.
